(19) United States
(12) Patent Application Publication
(10) Pub. No.: US 2012/0277444 A1
(43) Pub. Date: Nov. 1, 2012

(54) SYNTHESIS OF HYDROXYALKYL AMIDES FROM ESTERS

(76) Inventors: Shivkumar Mahadevan, Orange Park, FL (US); Kunisi Venkatasubban, Jacksonville, FL (US)

(21) Appl. No.: 13/415,235

(22) Filed: Mar. 8, 2012

Related U.S. Application Data
(60) Provisional application No. 61/479,446, filed on Apr. 27, 2011.

(51) Int. Cl. C07C 231/02; C07D 307/68; C07D 209/18; C07C 303/22

(52) U.S. Cl. 548/502; 564/224; 562/106; 549/487; 564/186

(57) ABSTRACT

Hydroxyamides are synthesized from esters. A process of making hydroxyalkyl amides comprises: reacting an ester with a hydroxyalkyl amine having the formula H₂N—R₃—OH wherein R₃ is a substituted or unsubstituted C2 to C5 alkyl, in the presence of a catalyst in an anhydrous solution to form the hydroxyalkyl amides. Monomers suitable for formation of polymeric articles can utilize these hydroxyamides.

### RELATED APPLICATIONS

This application claims priority to U.S. Provisional Patent Application No. 61/479,446 filed on Apr. 27, 2011 entitled SYNTHESIS OF HYDROXYALKYL AMIDES FROM ESTERS, the contents of which are incorporated by reference.

### TECHNICAL FIELD

The present invention relates to processes for synthesizing hydroxyamides from esters.

### BACKGROUND

Mild aminolysis of esters is a desirable transformation. Amide derivatives with additional functional groups allow for further derivatization of the compounds providing opportunities to prepare new synthetic target molecules. Such reactions provide access to compounds that can be used as monomers for various polymeric articles such as contact lenses. Hydroxyalkyl amido compounds are one such example, where the hydroxyl group of the compound may be further functionalized to form aldehydes, carboxylic acids, esters, etc. Some examples of monomers that may be prepared by functionalization of the hydroxyl groups are methacrylates, acrylates, and olefins.

Aminolysis of esters, however, is not generally a facile reaction. Usually harsh reaction conditions are required to achieve this transformation: such as, catalysis by strong bases or metals under elevated temperatures. Such conditions then generally lead to unwanted side reactions in the presence of sensitive functional groups.

There is an ongoing need in the art to provide efficient processes that can produce amides with desirable functional groups for use in syntheses of polymeric materials for biomedical articles, for example, ophthalmic devices such as contact lenses.

### SUMMARY

Provided are chemical processes for synthesizing hydroxyalkyl amides from esters. Methods of using the same are also provided. In a first aspect, a process of making hydroxyalkyl amides comprises: reacting an ester with a hydroxyalkyl amine having the formula H₂N—R₃—OH wherein R₃ is a substituted or unsubstituted C2 to C5 alkyl, in the presence of a catalyst in an anhydrous solution to form the hydroxyalkyl amides. Other aspects include preparing monomers suitable for polymeric articles utilizing hydroxyamides.

### BRIEF DESCRIPTION OF THE DRAWINGS

FIGS. 1A, 1B, 1C, and 1D show NMR spectra of unreacted amines and resulting reaction mixtures under varying conditions according to an embodiment;

FIG. 2 shows NMR spectra of resulting reaction mixtures of catalyzed versus uncatalyzed reactions according to an embodiment;

FIGS. 3A and 3B show NMR spectra of unreacted amines and resulting reaction mixtures according to an embodiment;

FIGS. 4A and 4B show NMR spectra of unreacted comparative amines and resulting reaction mixtures;

FIGS. 5A and 5B show NMR spectra of unreacted comparative amines and resulting reaction mixtures;

FIGS. 6A and 6B show NMR spectra of unreacted comparative amines and resulting reaction mixtures;

FIGS. 7A and 7B show NMR spectra of resulting reaction mixtures according to an embodiment;

FIGS. 8A and 8B show NMR spectra of resulting reaction mixtures according to an embodiment;

FIGS. 9A and 9B show NMR spectra of resulting reaction mixtures according to an embodiment;

FIGS. 10A, 10B, 10C, and 10D show NMR spectra of an unreacted ester and resulting reaction mixtures;

FIGS. 11A and 11B show NMR spectra of an unreacted esters and resulting reaction mixtures;

FIGS. 12A, 12B, 12C, and 12D show NMR spectra of an unreacted ester and resulting reaction mixture; and

FIGS. 13A and 13B show NMR spectra of an unreacted comparative ester and resulting reaction mixtures.

### DETAILED DESCRIPTION

Provided are processes of making and using hydroxyalkyl amides. The processes, in general terms, include reacting an ester with a hydroxyalkyl amine in the presence of a catalyst to form the hydroxyalkyl amides. The catalyst can be a heterogeneous catalyst, meaning that a solid catalyst is dispersed in the liquid reaction media. The catalyst for the present processes can comprise an alkali metal salt in an anhydrous solution. Polar protic solvents such as methanol and ethanol are preferable for such reactions, though the reactions may proceed in solvents such as dimethyl sulfoxide (DMSO) or N,N-dimethylformamide (DMF). Generally, the reaction conditions are mild. Appreciable amounts of hydroxyalkyl amides can be formed after twenty-four hours under ambient conditions. Derivatives of volatile esters are easily obtained by filtration, followed by evaporation of the volatile components at reduced pressure, allowing for energy and labor efficient manufacturing regimes.

Transesterification of esters with alcohols occurs under mild conditions and may even be performed using heterogeneous catalysis in alcoholic media. It has been discovered that hydroxyalkyl amino compounds may be used to form hydroxyalkyl amides from esters under mild conditions in the presence of, for example, methanolic sodium carbonate. Without intending to be bound by theory, the proposed mechanism involves initial transesterification, followed by intramolecular rearrangement to produce the desired amides. A representative scheme is depicted in Formula (I).

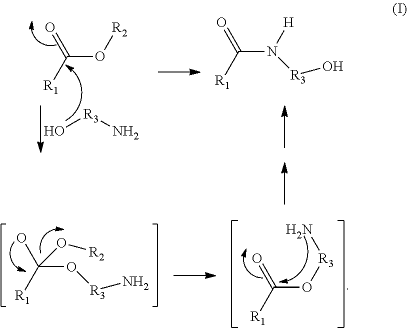

The reactions are typically complete in about 24 hours under ambient conditions. Yields of hydroxyalkyl amides are essentially quantitative under anhydrous conditions, and the desired products are recovered by filtering out the heterogeneous catalyst and evaporating all the volatiles at reduced pressure. In this way, the final product is obtained under mild conditions without the need for further physical separation by distillation or fractionation. In addition, the solid catalyst can be dried and re-used in subsequent reactions.

In a first aspect, provided are processes of making hydroxyalkyl amides comprising: reacting an ester with a hydroxyalkyl amine having the formula H₂N—R₃—OH wherein R₃ is a substituted or unsubstituted C2 to C5 alkyl, in the presence of a catalyst in an anhydrous solution to form the hydroxyalkyl amides. Reference to “substituted” means that a hydrogen atom has been removed from the alkyl and replaced with anything other than a hydrogen atom, typically an alkyl, an aryl, and the like. Reference to “unsubstituted” means no H atom has been removed.

The ester can have the formula R₁—CO₂—R₂. R₂ can comprise a substituted or unsubstituted C1 to C10 alkyl group which is not substantially sterically hindered. Specifically, wherein R₂ can comprise a C1 to C10 (or C1 to C5 or even C1 to C3) substituted or unsubstituted alkyl. In a specific embodiment, R₂ comprises an unsubstituted C1 to C5 primary alkyl.

Generally, R₁ can be non-nucleophilic such that it does not compete with the transesterification and rearrangement reaction (i.e., R₁ does not contain the combination of a hydroxyl group and an amine). That is, R₁ can be selected from the group consisting of hydrocarbons, alcohols, carboxylic acids, ethers, phosphates, sulfonates, and combinations thereof. In specific embodiments, the ester can comprises ethyl acetate or the methyl ester of cysteic acid, or combinations thereof. The structure of R₁ can provide as many functional groups as is practical and needed to form resulting hydroxyalkyl amines that are useful for making compounds of desired functionality. Examples of such compound are monomers that are useful for biomedical devices, such as contact lenses, are biocompatible, hydrophilic (sphere of hydration), resistant to deposits. Monomers that are surfactants (ionic or non-ionic) are particularly useful. Siloxanes, aldehydes, alkyl halides, ketones, and other functional groups that are reactive towards amines and alcohols are generally incompatible with this process.

In one or more embodiments, the hydroxyalkyl amine comprises 3-aminopropanol or 2-aminoethanol, or combinations thereof.

Other embodiments provide that the catalyst is a heterogeneous catalyst. The catalyst can comprise an alkali metal salt. The alkali metal salt can comprise a carbonate, an alkoxide, or combinations thereof. The alkali metal salt can comprise sodium (Na+), potassium (K+), lithium (Li+), cesium (Cs+) ions, or combinations thereof. The alkali metal salt can comprise sodium carbonate, lithium carbonate, or combinations thereof. Other embodiments provide that the catalyst comprises N-alkyl ammonium carbonate or N-alkyl ammonium alkoxide.

As desired, the anhydrous solution can comprise methanol, ethanol, propanol, or combinations thereof.

Detailed embodiments provide that the hydroxyalkyl amides are selected from the group consisting of 3-hydroxypropyl acetamide, 2-hydroxyethyl acetamide, hydroxypropyl and hydroxyethyl amides of cysteic acid and its derivatives, hydroxypropyl and hydroxyethyl amides of ethylbenzoate and its derivatives, hydroxypropyl and hydroxyethyl amides of methyl-5-(3-hydroxyphenyl)-furan-2-carboxylate and its derivatives, hydroxypropyl and hydroxyethyl amides of methyl indole-3-acetate and its derivatives, or combinations thereof.

Another aspect provides methods of making monomers suitable for polymeric articles, the methods comprising: providing hydroxyamides and preparing a monomer mixture comprising the hydroxyalkyl amides. The hydroxyalkyl amides are particularly suitable for further derivatization of the compounds providing opportunities to prepare new synthetic target molecules. Such reactions provide access to compounds that can be used as monomers for various polymeric articles. Hydroxyalkyl amido compounds are one such example, where the hydroxyl group of the compound may be further functionalized to form aldehydes, carboxylic acids, esters, etc. Some examples of monomers that may be prepared by functionalization of the hydroxyl groups are methacrylates, acrylates, and olefins. Polymeric articles can include a medical device. As used herein, a “medical device” is any article that is designed to be used while either in or on mammalian tissues or fluid. Examples of these devices include but are not limited to catheters, implants, stents, and ophthalmic devices such as intraocular lenses and contact lenses.

In general terms, synthesis of hydroxyalkyl amides from esters reacted with hydroxyalkyl amines in the presence of a catalyst can be done at temperatures and pressures as desired and consistent with conventional manufacturing processes. While reactions can take place at room temperature (typically in the range of about 19-25° C.) without much need to go higher and ambient pressure, temperatures can be brought to higher ranges (about 25° C. to 80° C.) in order to accelerate the time to reaction completion. Anhydrous solutions are preferred for these syntheses.

When using volatile esters, the ester can be provided in an alcohol solution to which the amine, usually the limiting reagent, is added. The order of addition, however, can be changed to suit manufacturing needs. The unreacted ester may be removed by evaporation under reduced pressures. The amine can be added dropwise or all at once as needed. Mixing is done under conditions conducive to ensure adequate homogeneity of the mixture. Temperature can range widely and the reactions may be performed under ambient conditions of temperature and pressure, or in refluxing solvent if none of the reactants or products are thermally sensitive to the elevated temperatures. The ester may be used in excess, or as the limiting reagent, examples of both types are provided. The molar ratio of ester to amine can be on the order of the ranges of about 0.1:1 through 10:1.

When the ester is used as a limiting reagent, the desired product may be purified either by filtering the product mixture from the catalyst, evaporation of the volatile components, followed by extraction of the excess amine into an acidic aqueous layer, or by other methods known to those well versed in the art.

The endpoint of the synthesis is when sufficient hydroxyalkyl amide has been formed, preferably upon consumption of the entire limiting reagent. The reaction/synthesis mixture and resulting reaction mixture can be analyzed to determine the conversion and yields.

Nuclear Magnetic Resonance (NMR) Spectra

NMR spectra were obtained using a Bruker Advance 300 MHz instrument and the raw data were processed using NMR utilities Transform Software (NUTS) developed by Acorn NMR, Inc. Livermore, Ca.

Mass Spectrometry

Molecular weights for the component(s) for each product/product mixture were obtained using a Thermo LCQ Advantage Ion Trap LC-MS system. Separation and analysis were achieved by reverse phase HPLC using a Phenomenex Synergi column (50×4.6 mm×4 u) with an acetonitrile/water (0.1% TFA) gradient followed by electrospray ionization. Structures were confirmed by the presence of the protonated molecular ion (M+H⁺).

Before describing several exemplary embodiments of the invention, it is to be understood that the invention is not limited to the details of construction or process steps set forth in the following description. The invention is capable of other embodiments and of being practiced or being carried out in various ways.

### EXAMPLES

Reactions of various amines (hydroxylated and unhydroxylated) with certain esters were investigated.

### Example 1

Ethyl acetate in the amount of 5 g (56.8 mMole) was reacted with 20 mMoles of 2-aminoethanol in the presence of methanolic sodium carbonate under varying sets of conditions: 24 hours at ambient conditions and both 24 and 36 hours under reflux at 60° C. The mixtures were cooled as needed to room temperature, and the sodium carbonate was removed by filtration. Reaction products were obtained by evaporation of unreacted ethyl acetate and methanol under reduced pressure. Yield of the resulting amide, 2-hydroxyethyl acetamide, based on starting material recovered was 98% at 73% conversion at ambient conditions after 24 hours. At 60° C., yields of the resulting amide, 2-hydroxyethyl acetamide, based on starting material recovered were 95% at 90% conversion after 24 hours and 95% at 100% conversion after 36 hours.

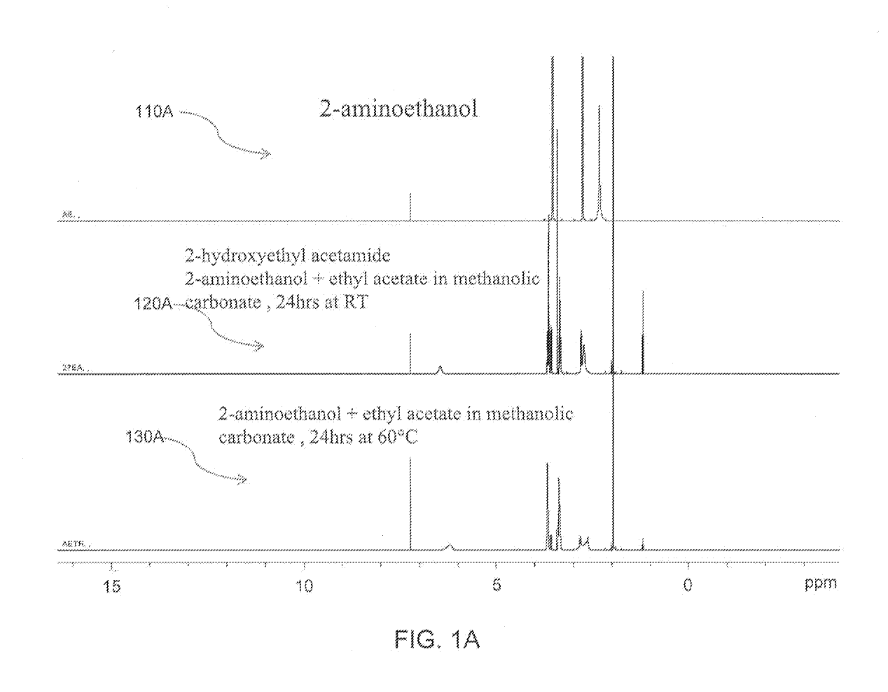

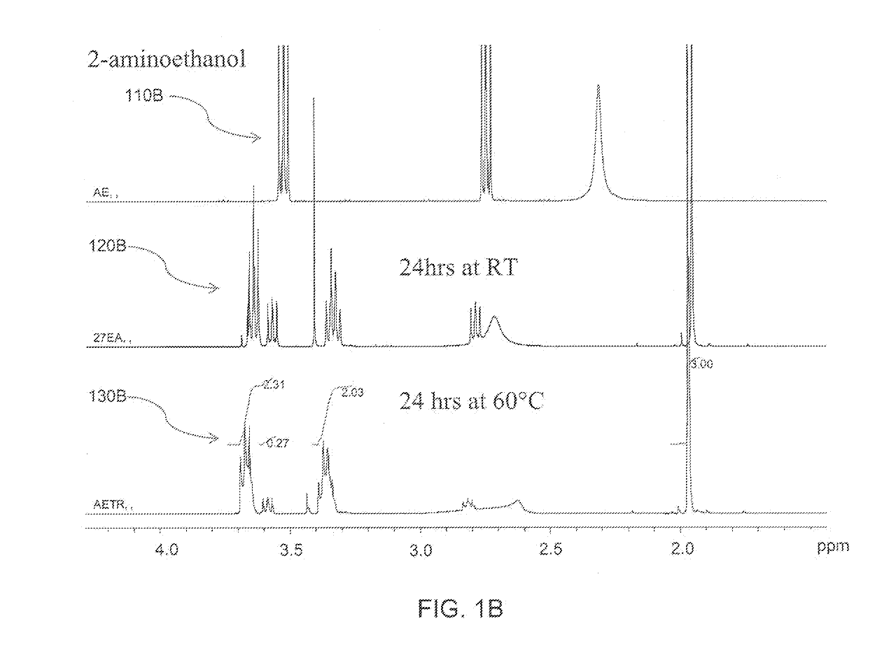

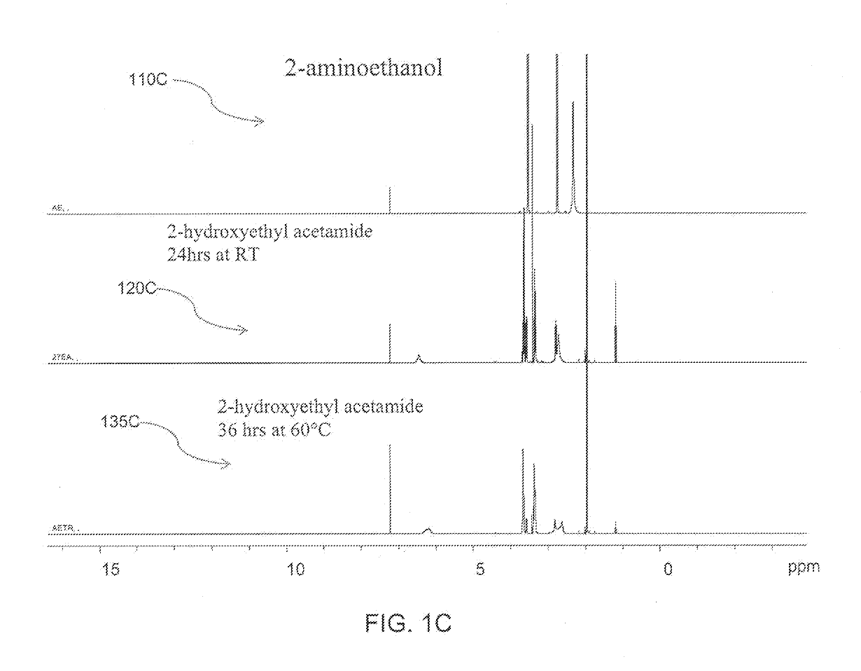

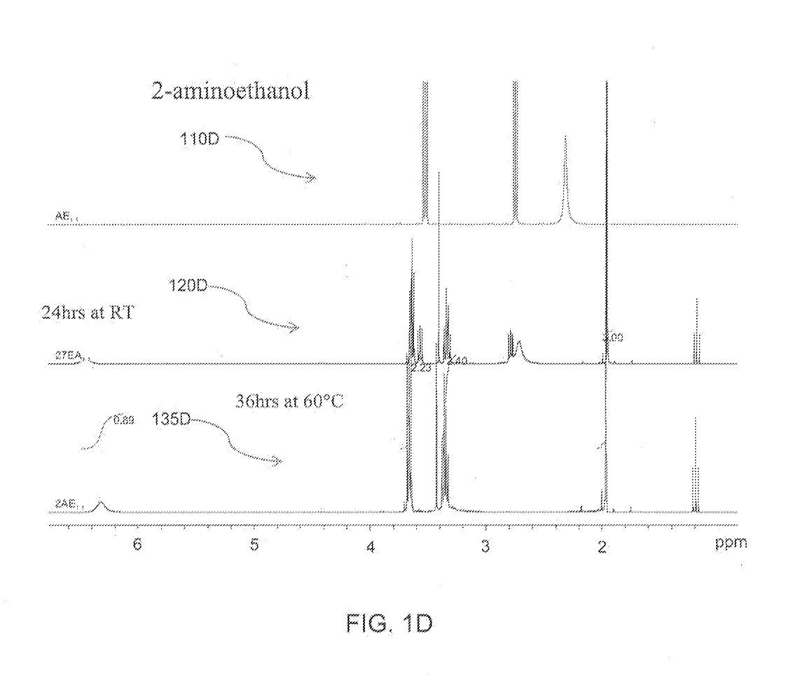

NMR spectra for Example 1 are provided in FIGS. 1A, 1B, 1C, and 1D, where spectra 110A, 110B, 110C, and 110D are 2-aminoethanol; spectra 120A, 120B, 120C, and 120D are the reaction mixture after 24 hours at ambient conditions. Spectra 130A and 130B are the resulting reaction mixture after 24 hours at 60° C. Spectra 135C and 135D are the resulting reaction mixture after 36 hours at 60° C. Product structure of 2-hydroxyethyl acetamide was also confirmed by mass spectrometry.

### Example 2

### Comparative

Ethyl acetate in the amount of 5 g (56.8 mMole) was reacted with 20 mMoles of 2-aminoethanol in the absence of a catalyst for 24 hours at ambient conditions. Yield of the resulting amide, 2-hydroxyethyl acetamide, based on starting material recovered was 98% at 37% conversion at ambient conditions after 24 hours.

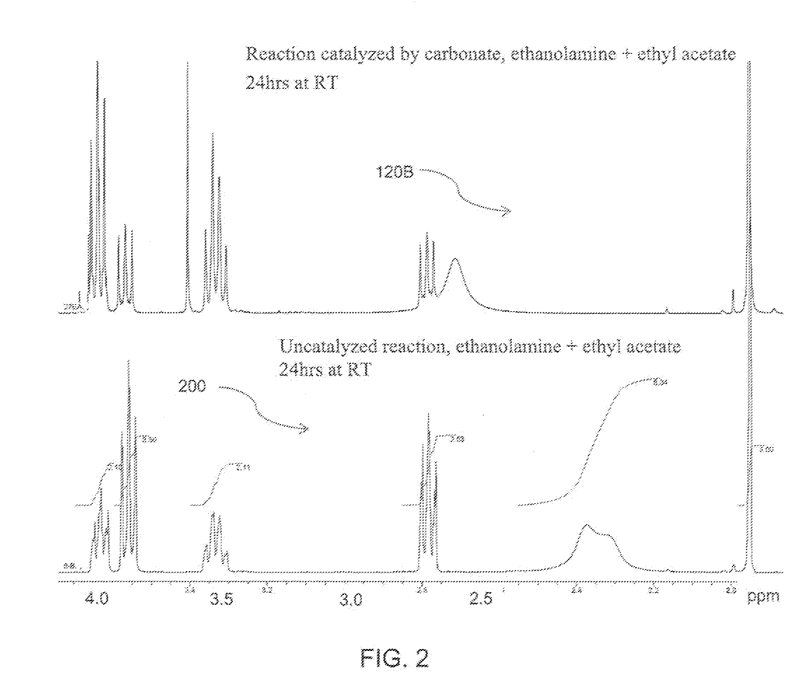

NMR spectra for Example 2 are provided in FIG. 2, where spectrum 120B is the reaction mixture after 24 hours at ambient conditions (which was in FIG. 1B). Spectrum 200 is the resulting reaction mixture after 24 hours at ambient conditions in the absence of a catalyst. Product structure of 2-hydroxyethyl acetamide was also confirmed by mass spectrometry.

### Example 3

Ethyl acetate in the amount of 5 g (56.8 mMole) was reacted with 20 mMoles of 3-aminopropanol in the presence of methanolic sodium carbonate under varying sets of conditions: 24 hours at both ambient conditions and under reflux at 60° C. The mixtures were cooled as needed to room temperature, and the sodium carbonate was removed by filtration. Reaction products were obtained by evaporation of unreacted ethyl acetate and methanol under reduced pressure. Yield of the resulting amide, 2-hydroxypropyl acetamide, based on starting material recovered was 91% at 74% conversion at ambient conditions after 24 hours. At 60° C., yield of the resulting amide, 2-hydroxypropyl acetamide, based on starting material recovered was 92% at 90% conversion after 24 hours.

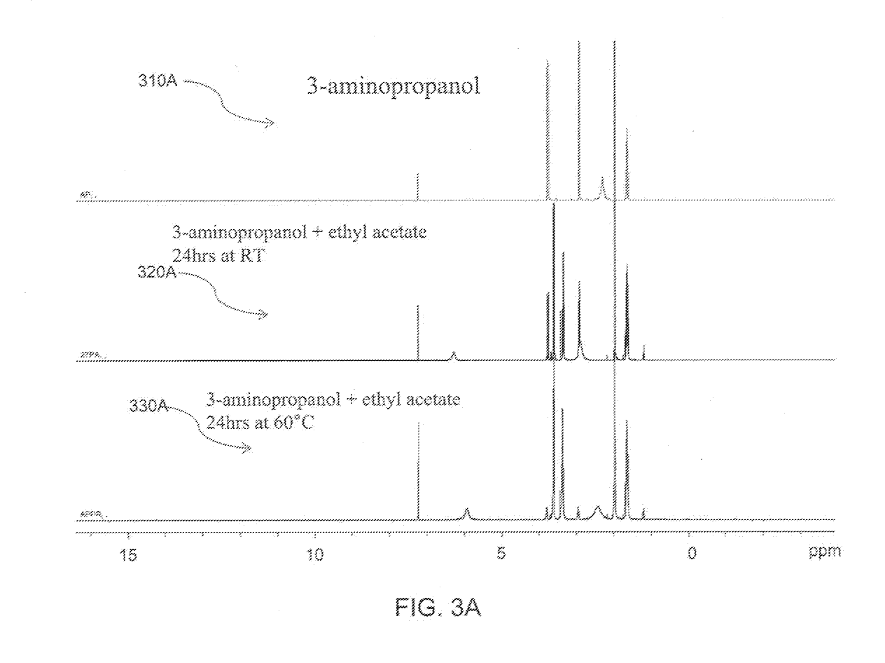

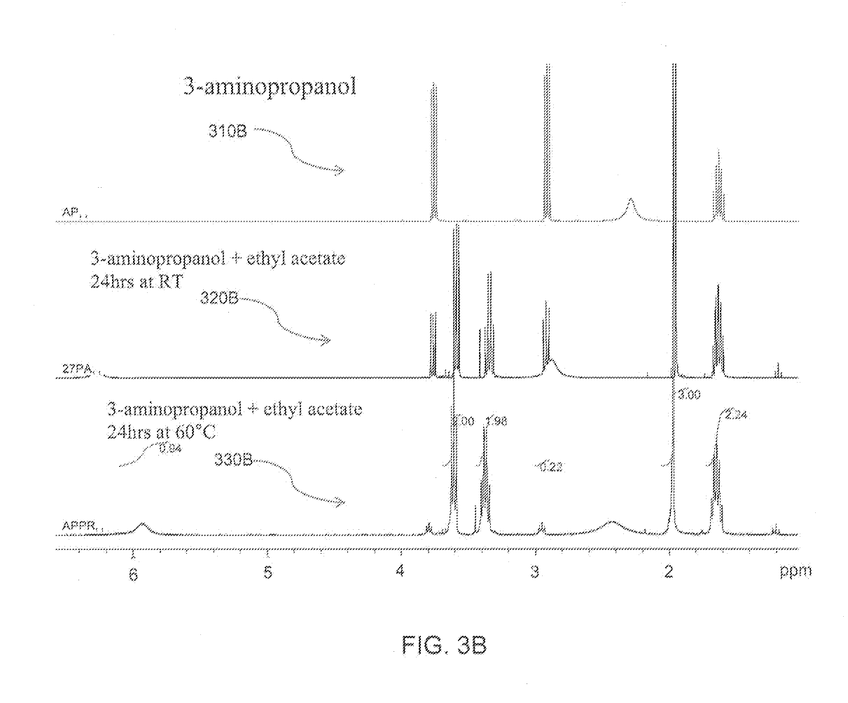

NMR spectra for Example 3 are provided in FIGS. 3A and 3B, where spectra 310A and 310B are 3-aminopropanol; spectra 320A and 320B are the reaction mixture after 24 hours at ambient conditions. Spectra 330A and 330B are the resulting reaction mixture after 24 hours at 60° C. Product structure of 2-hydroxypropyl acetamide was also confirmed by mass spectrometry.

### Example 4

### Comparative

Ethyl acetate in the amount of 5 g (56.8 mMole) was reacted with 20 mMoles of N-allylamine in the presence of methanolic sodium carbonate for 24 hours both at ambient conditions and under reflux at 60° C. The mixtures were cooled as needed to room temperature, and the sodium carbonate was removed by filtration. Reaction products were obtained by evaporation of unreacted ethyl acetate and methanol under reduced pressure. Isolated yields of the resulting amide, allyl acetamide, were 22% at ambient and 50% at 60° C.

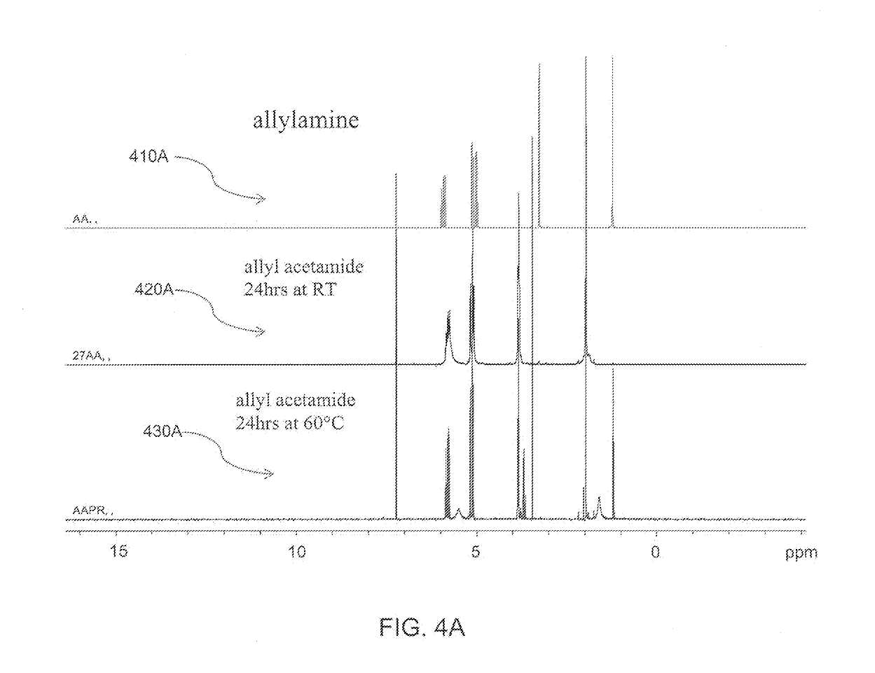

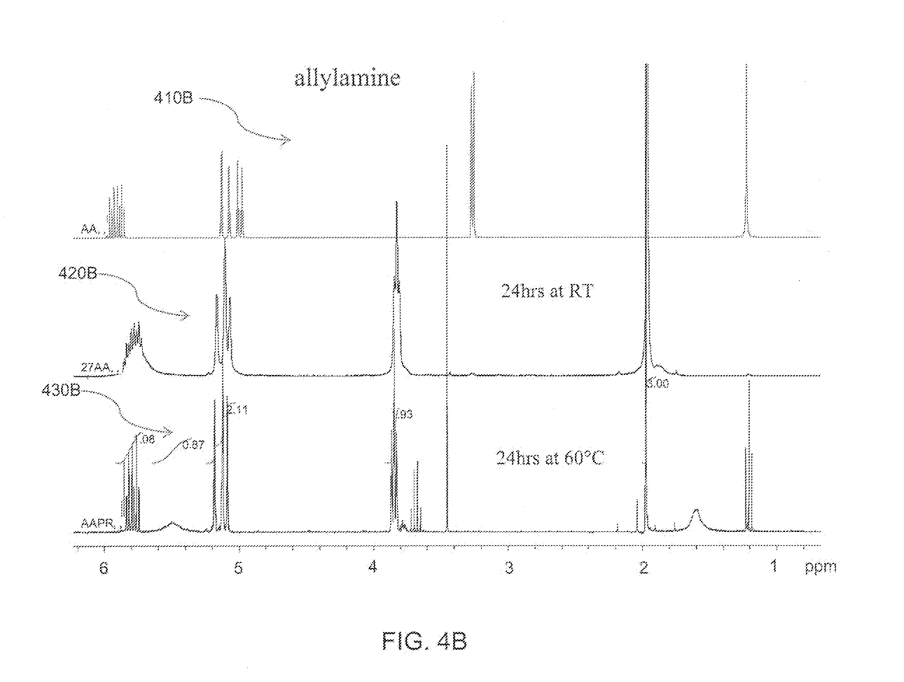

NMR spectra for Example 4 are provided in FIGS. 4A and 4B, where spectra 410A and 410B are N-allylamine, spectra 420A and 420B are the reaction mixture after 24 hours at ambient conditions, and spectra 430A and 430B are the resulting reaction mixture after 24 hours at 60° C. Product structure of allyl acetamide was also confirmed by mass spectrometry.

### Example 5

### Comparative

Ethyl acetate in the amount of 5 g (56.8 mMole) was reacted with 20 mMoles of cysteamine in the presence of methanolic sodium carbonate at ambient conditions and under reflux at 60° C. The mixtures were cooled as needed to room temperature, and the sodium carbonate was removed by filtration. Reaction products were obtained by evaporation of unreacted ethyl acetate and methanol under reduced pressure. In this example, there was no appreciable yield for any amide-based product at either temperature.

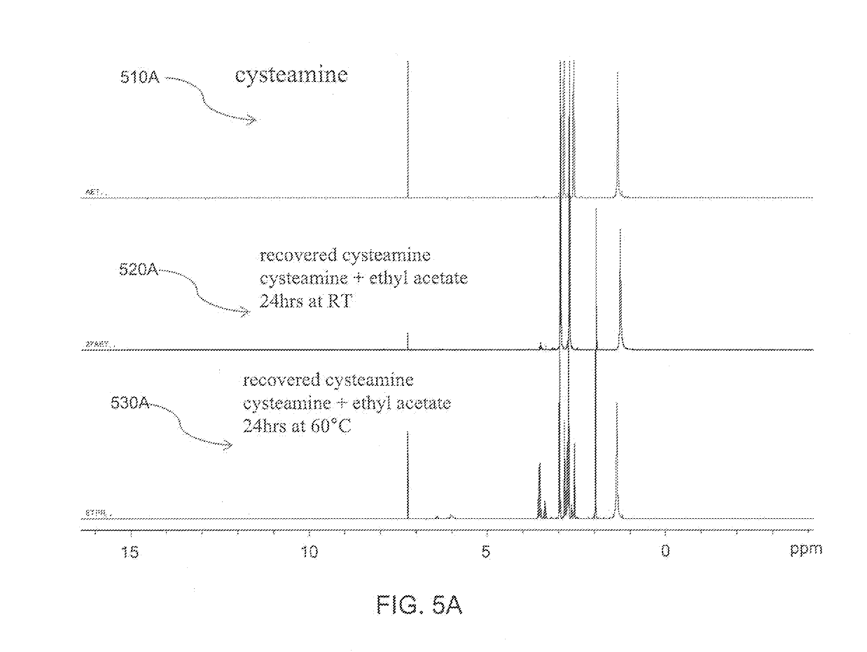

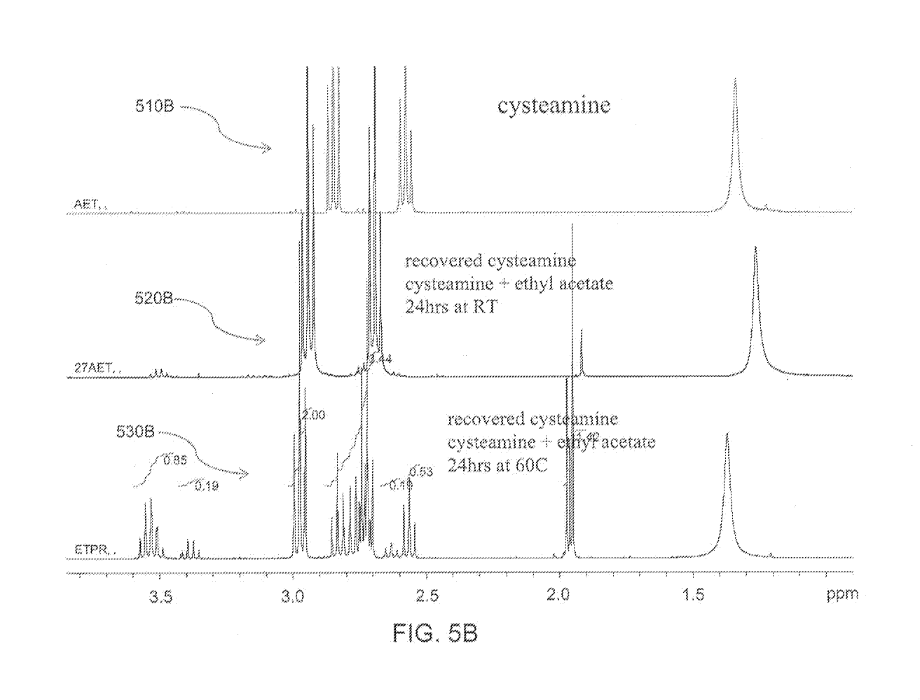

NMR spectra for Example 5 are provided in FIGS. 5A and 5B, where spectra 510A and 510B are neat cysteamine, spectra 520A and 520B are the reaction mixture after 24 hours at ambient conditions, and spectra 530A and 530B are the resulting reaction mixture after 24 hours at 60° C. Some unknown derivatives containing acetyl signals showed up in the 530B spectra of FIG. 5B.

### Example 6

### Comparative

Ethyl acetate in the amount of 5 g (56.8 mMole) was reacted with 20 mMoles of propylamine (also referred to as aminopropane) in the presence of methanolic sodium carbonate at ambient conditions and under reflux at 60° C. The mixtures were cooled as needed to room temperature, and the sodium carbonate was removed by filtration. Reaction products were obtained by evaporation of unreacted ethyl acetate and methanol under reduced pressure. Isolated yields of the resulting amide, N-propyl acetamide, were 25% at ambient and 65% at 60° C.

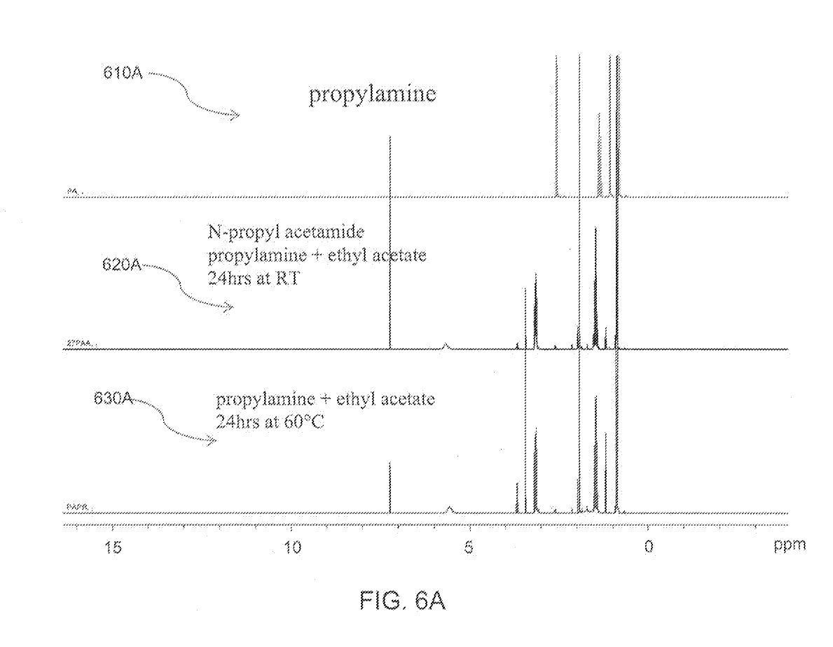

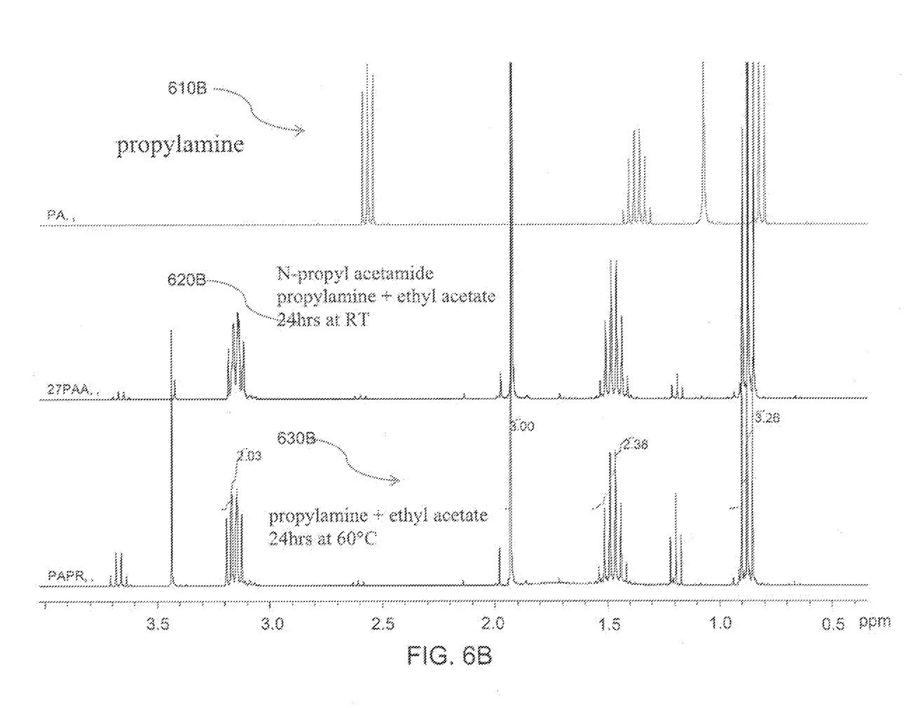

NMR spectra for Example 6 are provided in FIGS. 6A and 6B, where spectra 610A and 610B are propylamine, spectra 620A and 620B are the reaction mixture after 24 hours at ambient conditions, and spectra 630A and 630B are the resulting reaction mixture after 24 hours at 60° C. Product structure of N-propyl acetamide was also confirmed by mass spectrometry.

### Example 7

### Analysis

It can be seen that the yields of amides from the reactions of the hydroxyalkyl amines (Examples 1 and 3) were significantly greater than those from the non-hydroxylated amines (Comparative Examples 4-6). That is, the hydroxyalkyl amines studied (3-aminopropanol and 2-aminoethanol) provided quantitative yields of the desired amido derivatives. After 36 hours under reflux at 60° C., complete conversion of 2-aminoethanol to the corresponding acetamide was obtained.

In the cases of N-allylamine and aminopropane (N-propylamine), however, the yields of isolated products were significantly lower than in the case of hydroxyalkyl amines. In the case of 2-aminoethanethiol (cysteamine), little or no product was formed under the given conditions. Most of the starting material was recovered unchanged after the reaction.

As to the presence of a catalyst, the uncatalyzed reaction of Example 2 resulted in a yield of 98% at a conversion of 37% in contrast to the catalyzed reaction of Example 1 resulting in a yield of 98% at a conversion of 73%.

The non-hydroxylated amines were also studied at temperatures at reflux (nominal 60° C.) to determine if increased yields of amides could be obtained. Though increased yields of the desired products were obtained, significant differences in yield compared to the hydroxylated derivatives remained.

### Example 8

Esters showing low solubility in methanol also form hydroxyamides in high yields with hydroxyamino compounds. In this example, the methyl ester of cysteic acid was converted to its hydroxyethyl amide derivative in high yield under similar conditions as highly soluble esters (such as ethyl acetate). The proposed mechanism for this reaction is provided by Formula (II).

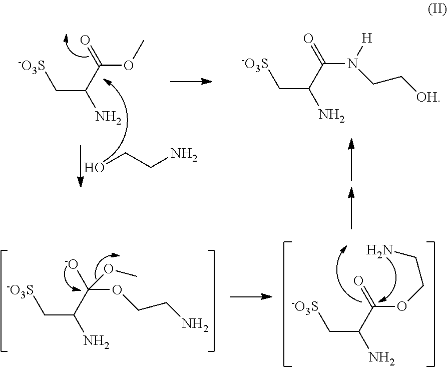

The methyl ester of cysteic acid (1.83 g, 0.01 mole) was heated overnight (approximately 18 hours) in 20 mL of methyl alcohol at 40° C. in the presence of 1.6 g (0.015 mole) of sodium carbonate and 1.22 g (0.02 mole) of 2-aminoethanol (ethanolamine). Upon cooling to room temperature, the solution was filtered and the product was precipitated by adding 60 mL of acetonitrile. The white solid was filtered, washed with acetonitrile and dried at 50° C. in a vacuum oven. The isolate had a structure of:

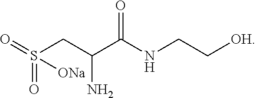

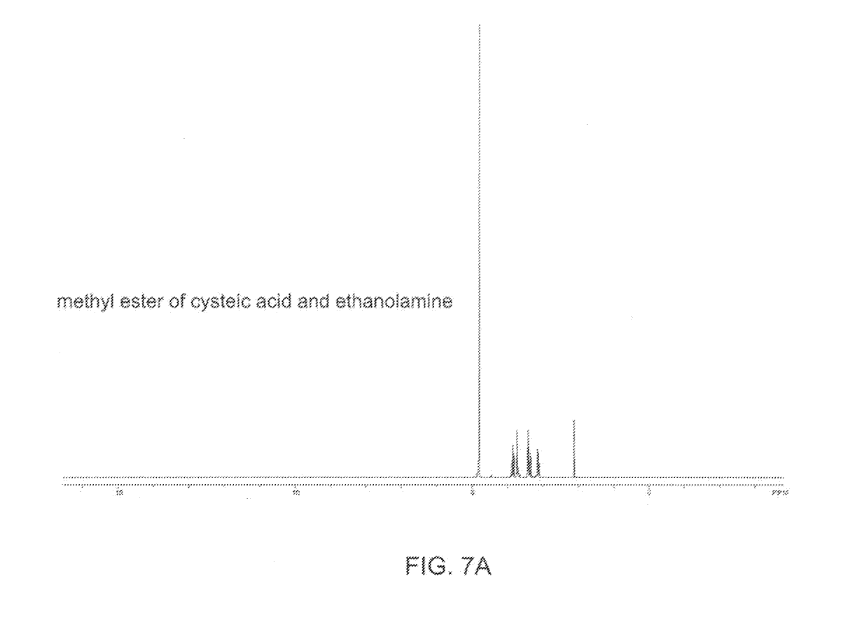

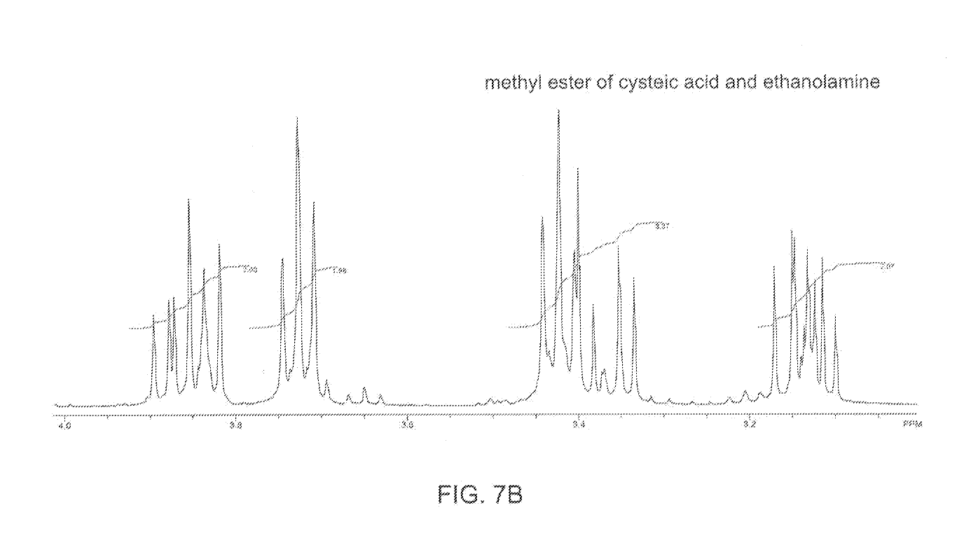

The NMR spectra for this Example are provided in FIGS. 7A and 7B.

### Example 9

In this example, an amide derivative of the methyl ester of cysteic acid was converted to its hydroxyethyl amide derivatives in high yield under similar conditions as highly soluble esters (such as ethyl acetate). Specifically, the decanamide derivative of the methyl ester cysteic acid was converted to its corresponding hydroxyethyl amide in high yields in refluxing methanolic carbonate over 48 hours. The sequence may be performed in one pot by converting the methyl ester to the desired amide in the presence of triethylamine and the required acid chloride, followed by the addition of carbonate and ethanolamine to effect the second transformation.

3.66 g of cysteic acid methyl ester (0.02 mole) and 5.05 g of triethylamine (0.05 mole) were dissolved in 60 mL of anhydrous methyl alcohol. Decanoyl chloride (4.77 g, 0.025 mole) was added to the solution in dropwise fashion while maintaining the temperature below 40° C. to convert the primary amine to the corresponding decanamide. The mixture was cooled to room temperature and 3.18 g (0.03 mole) of sodium carbonate and 1.83 g of ethanolamine (0.03 mole) were added. The mixture was then heated to reflux for 48 hours.

The volatile components were evaporated under reduced pressure and the residual solids were washed with ethyl acetate. The product can be further purified by soxhlet extraction using methyl alcohol, or by recrystallization in water, methyl alcohol, or any other appropriate solvent. The isolate had a structure of:

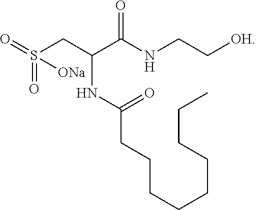

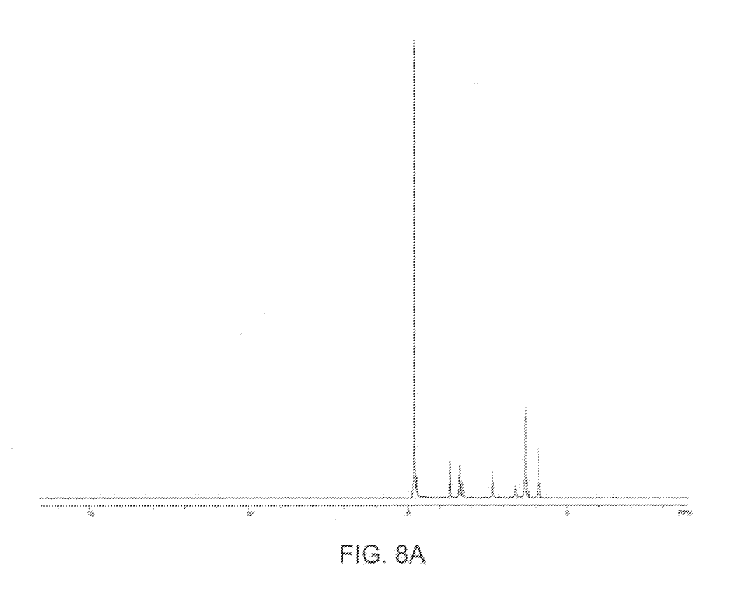

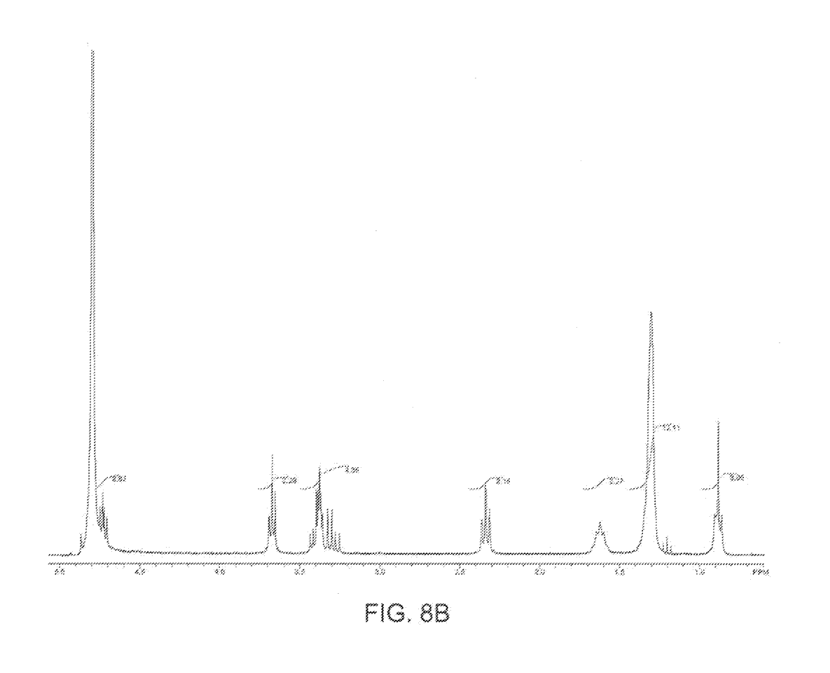

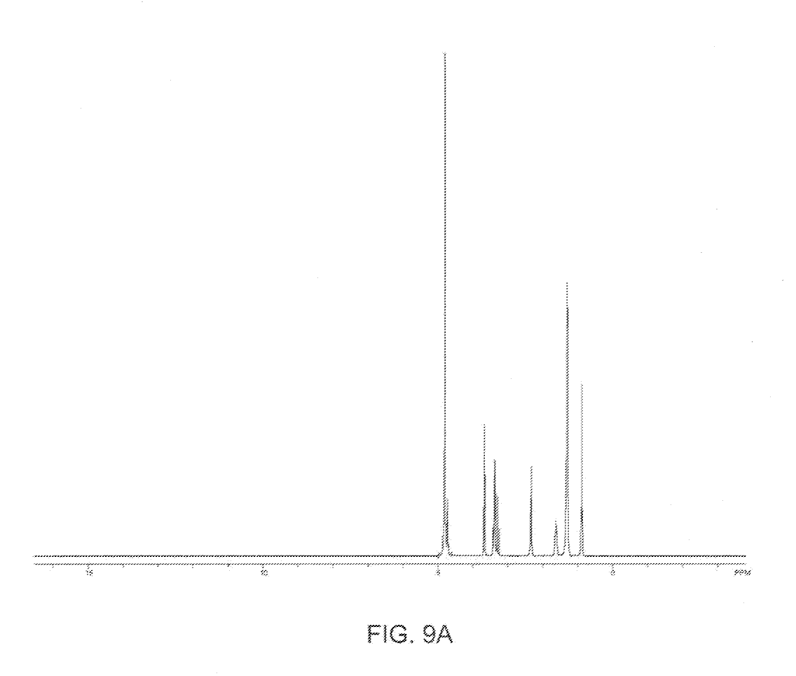

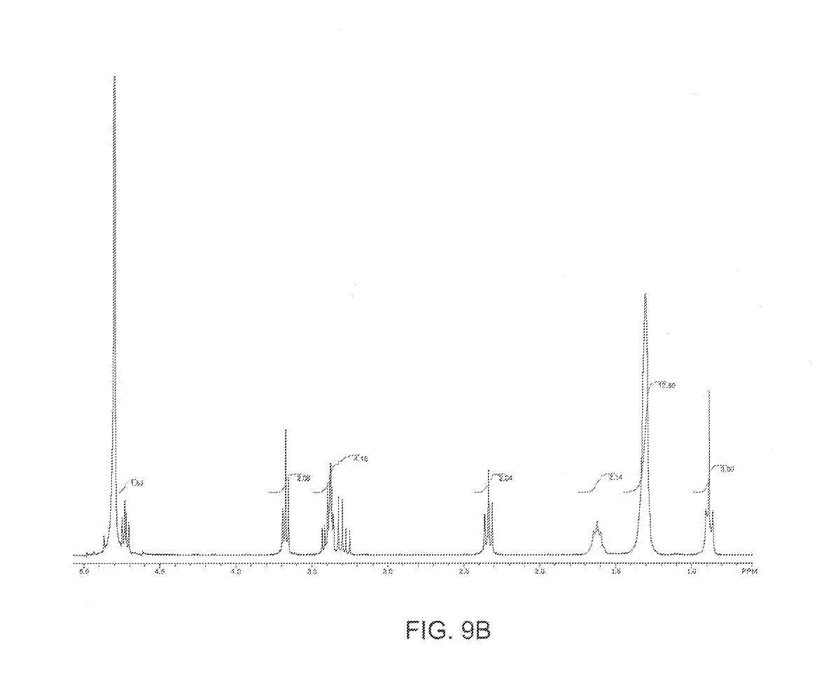

The NMR spectra for this Example as purified by soxhlet extraction are provided in FIGS. 8A and 8B. The NMR spectra for this Example as purified by recrystallization are provided in FIGS. 9A and 9B.

### Example 10

Methyl-5-(3-hydroxyphenyl)-furan-2-carboxylatein the amount of 2.3 mMole was reacted with 3.4 mMole ethanolamine in the presence of methanolic (7 mL of methanol) sodium carbonate (1 gram) under varying sets of conditions: 24 hours at both ambient conditions and at 60° C. The mixtures were cooled as needed to room temperature, and the sodium carbonate was removed by filtration. Reaction products were obtained by evaporation of methanol under reduced pressure. The ester and resulting amide are depicted as follows.

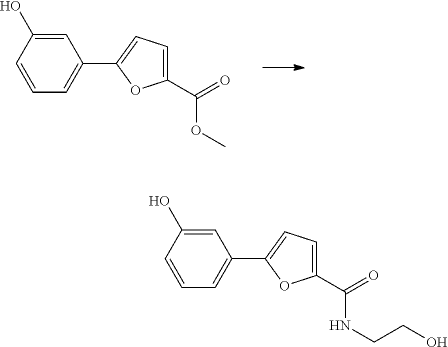

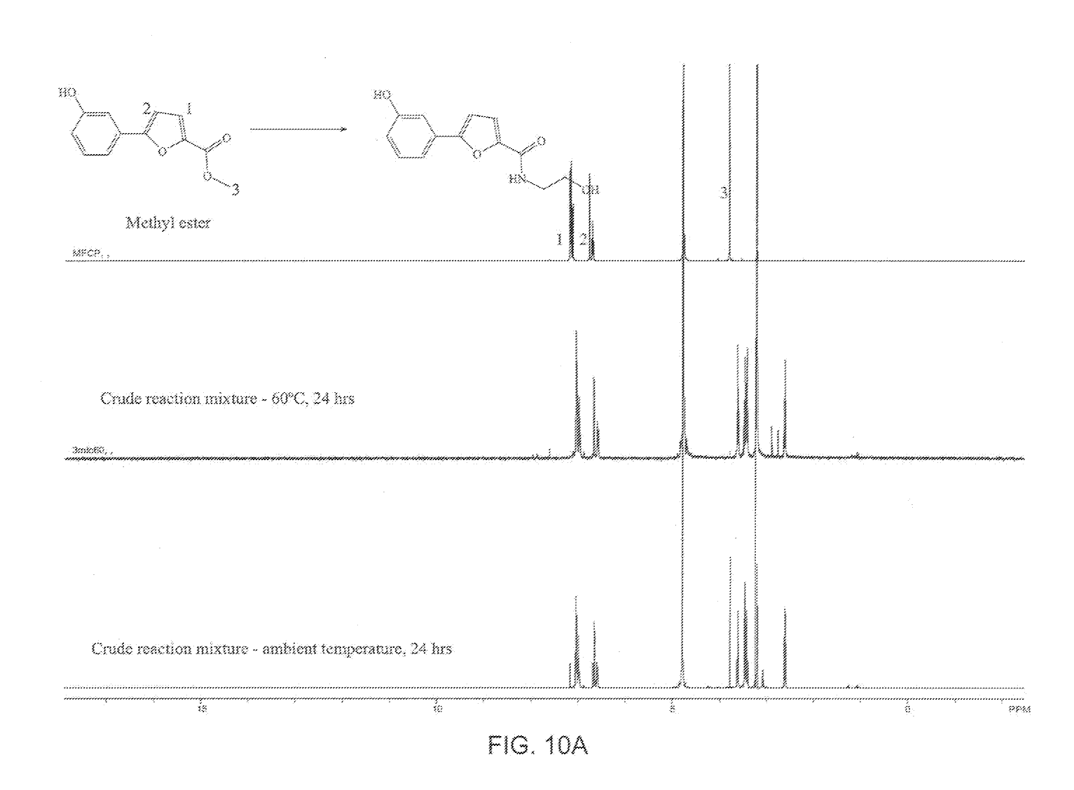

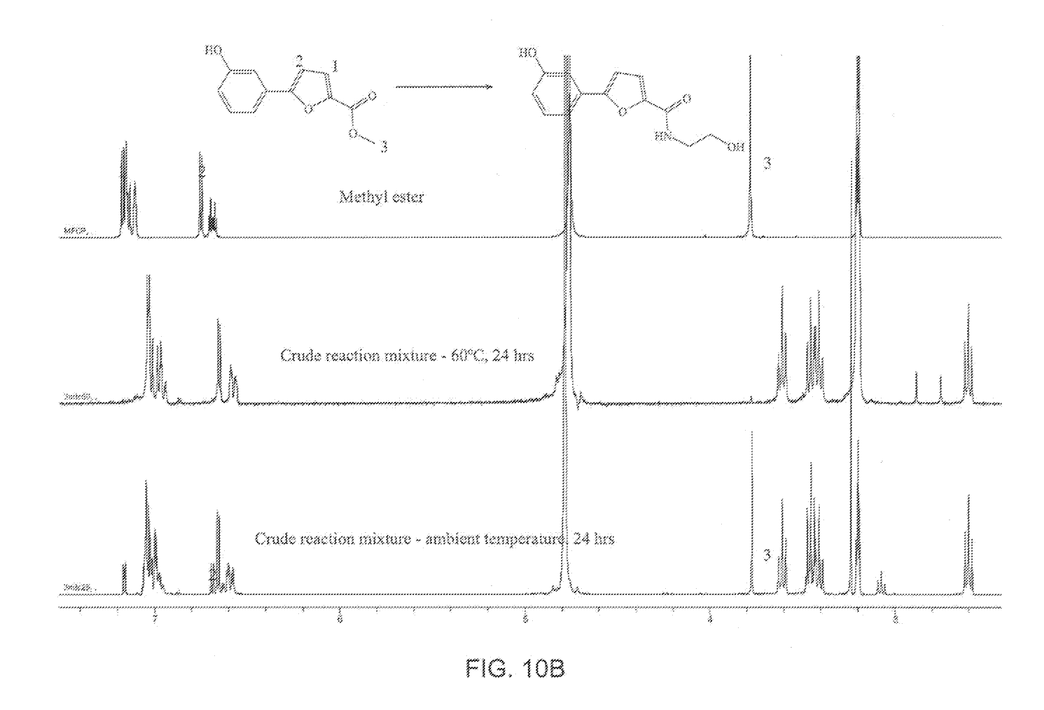

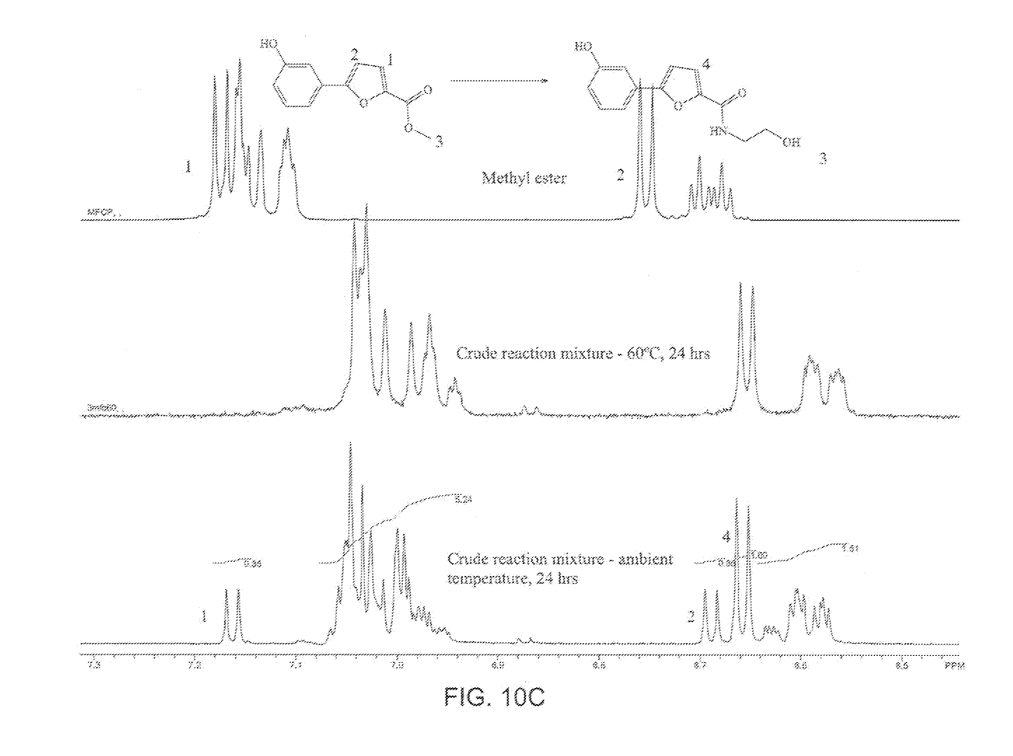

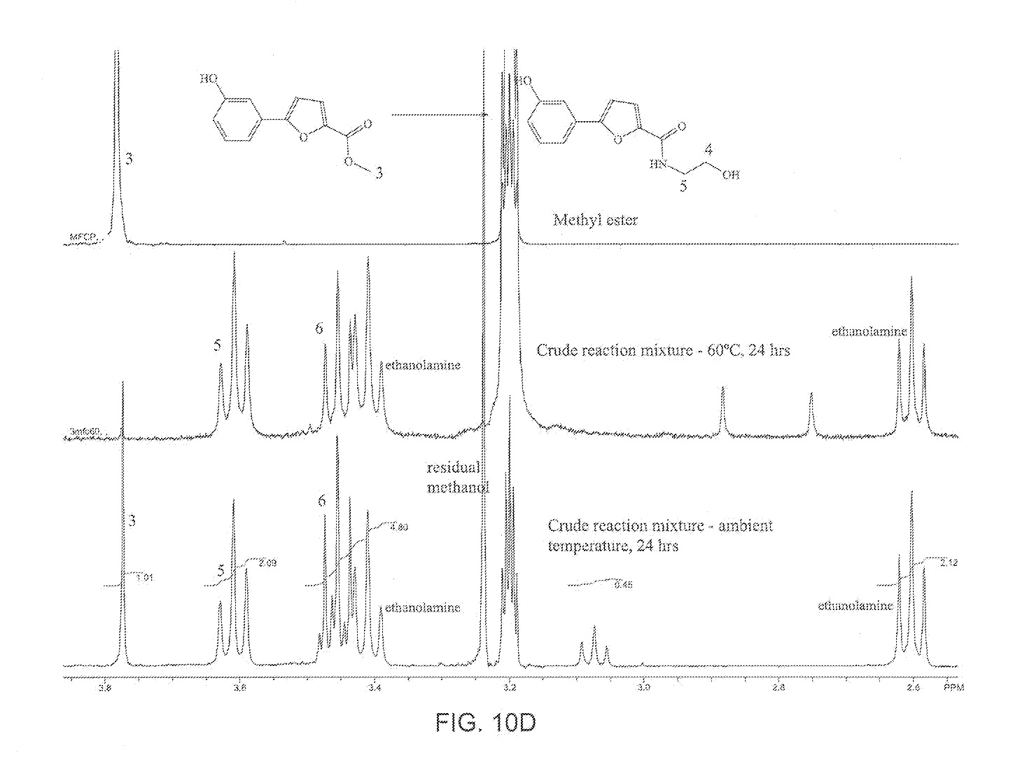

NMR spectra for Example 10 are provided in FIGS. 10A, 10 B, 10C, and 10D. The spectra of FIGS. 10C and 10D indicate approximately 75% (signals 2:4 of 10C) (signals 3:5 of 10D) conversion under ambient conditions and a quantitative reaction at 60° C.

### Example 11

Ethyl benzoate in the amount of 2.3 mMole was reacted with 3.4 mMole ethanolamine in the presence of methanolic (7 mL of methanol) sodium carbonate (1 gram) under varying sets of conditions: 24 hours at both ambient conditions and at 60° C. The mixtures were cooled as needed to room temperature, and the sodium carbonate was removed by filtration. Reaction products were obtained by evaporation of methanol under reduced pressure. The ester and resulting amide are depicted as follows.

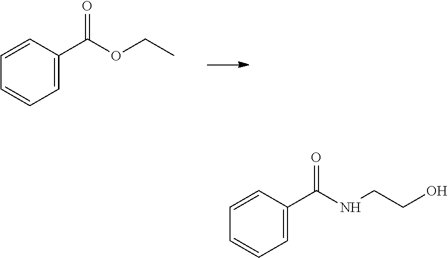

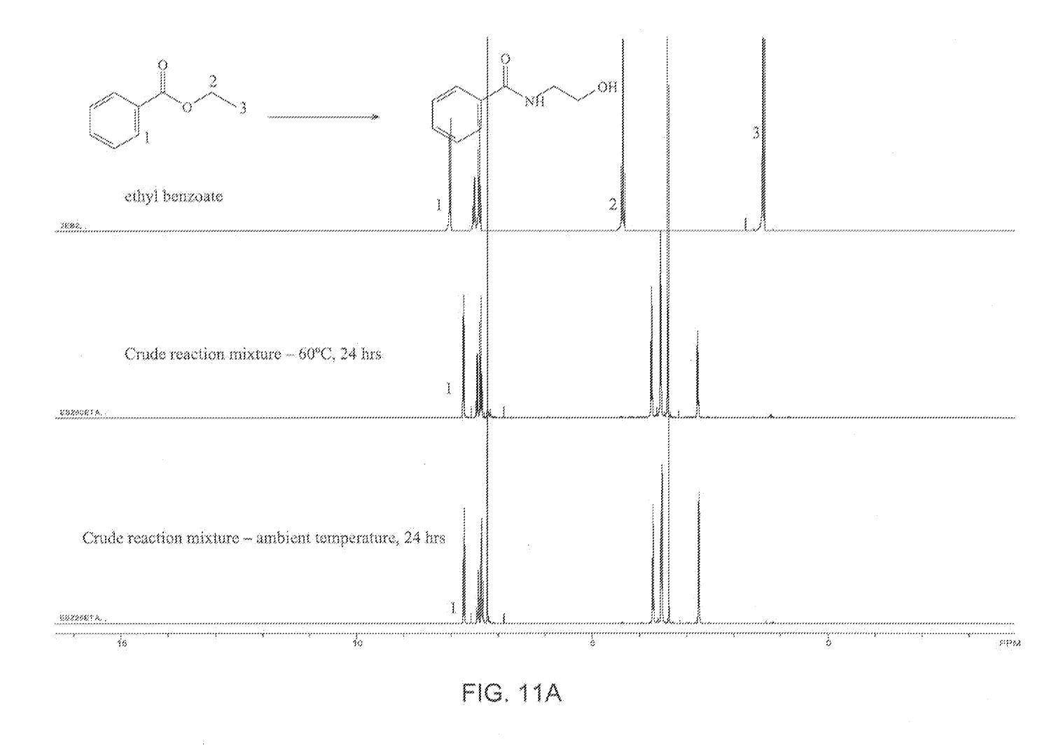

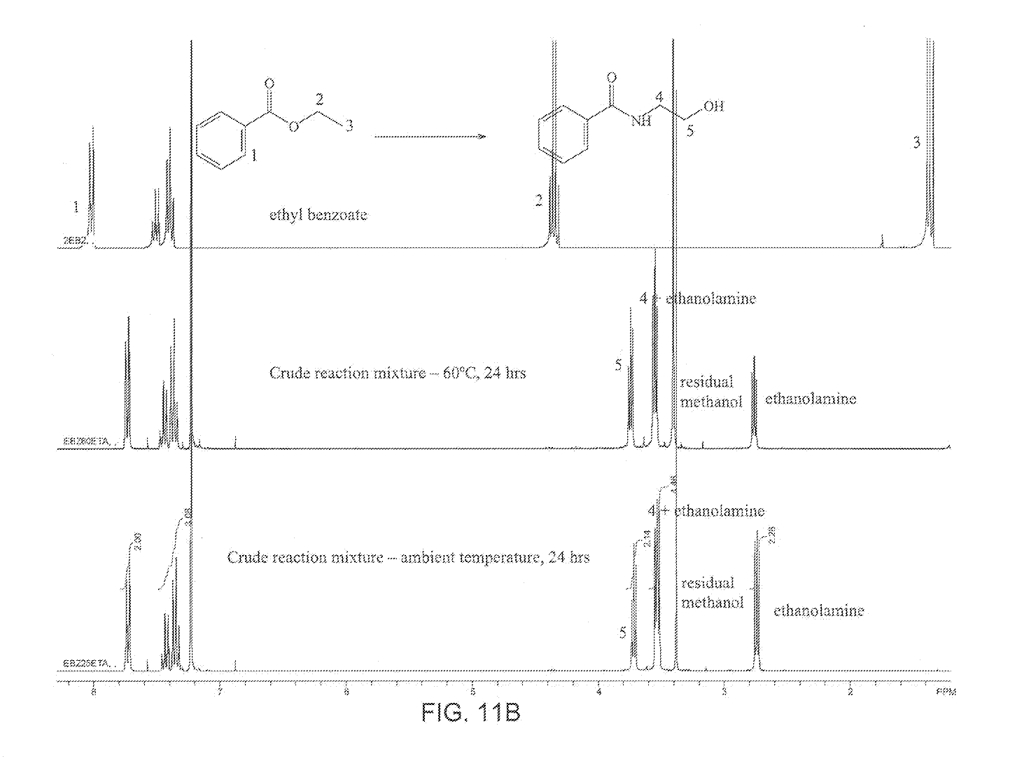

NMR spectra for Example 11 are provided in FIGS. 11A and 11B. The spectra show complete conversion of ethyl benzoate to the desired product under both ambient conditions and at 60° C.

### Example 12

Methyl indole-3-acetate in the amount of 2.25 mMole was reacted with 3.4 mMole ethanolamine in the presence of methanolic (7 mL of methanol) sodium carbonate (1 gram) under varying sets of conditions: 24 hours at both ambient conditions and at 60° C. The mixtures were cooled as needed to room temperature, and the sodium carbonate was removed by filtration. Reaction products were obtained by evaporation of methanol under reduced pressure. The ester and resulting amide are depicted as follows.

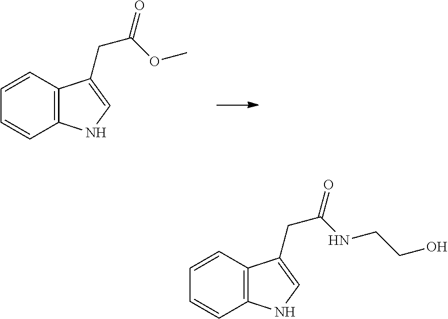

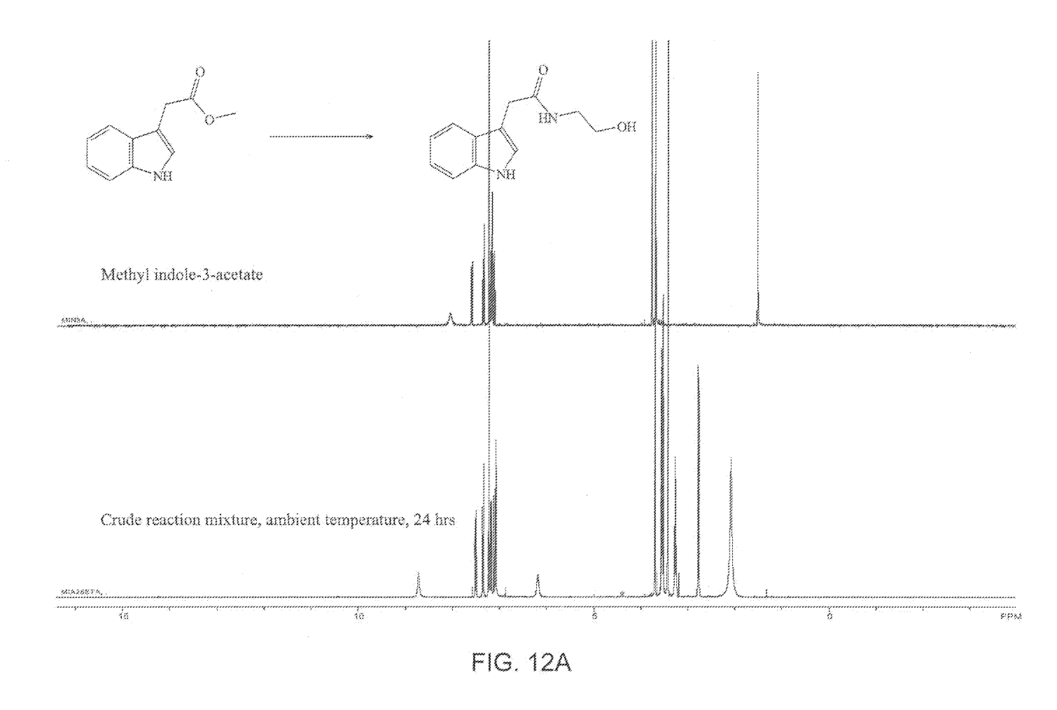

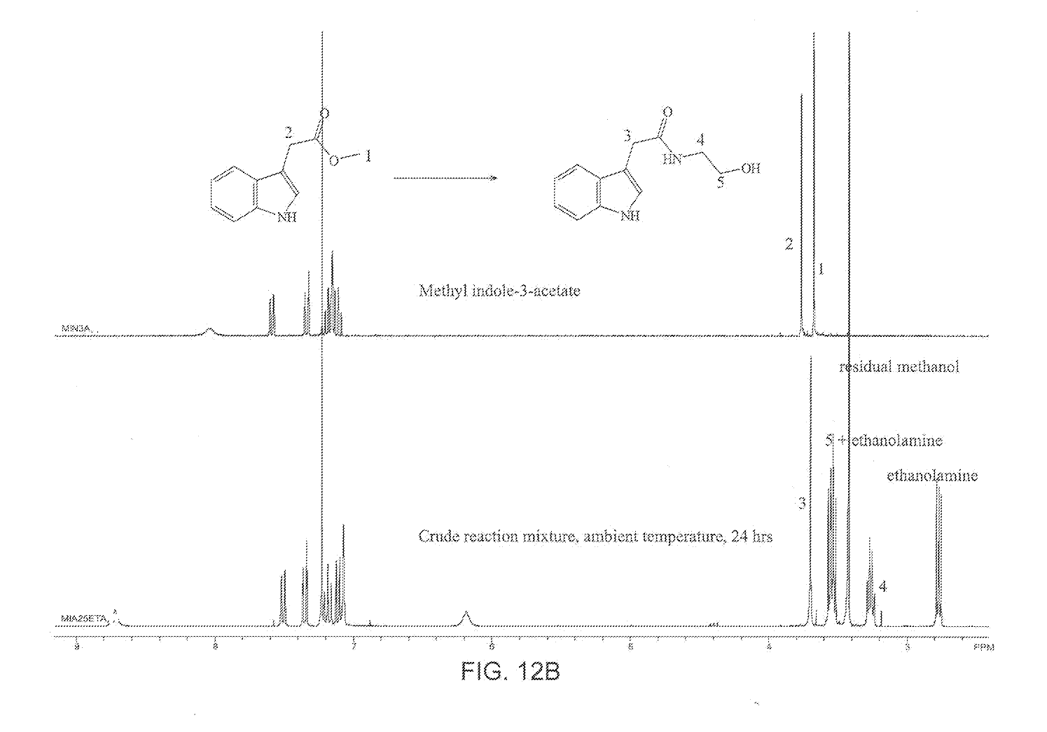

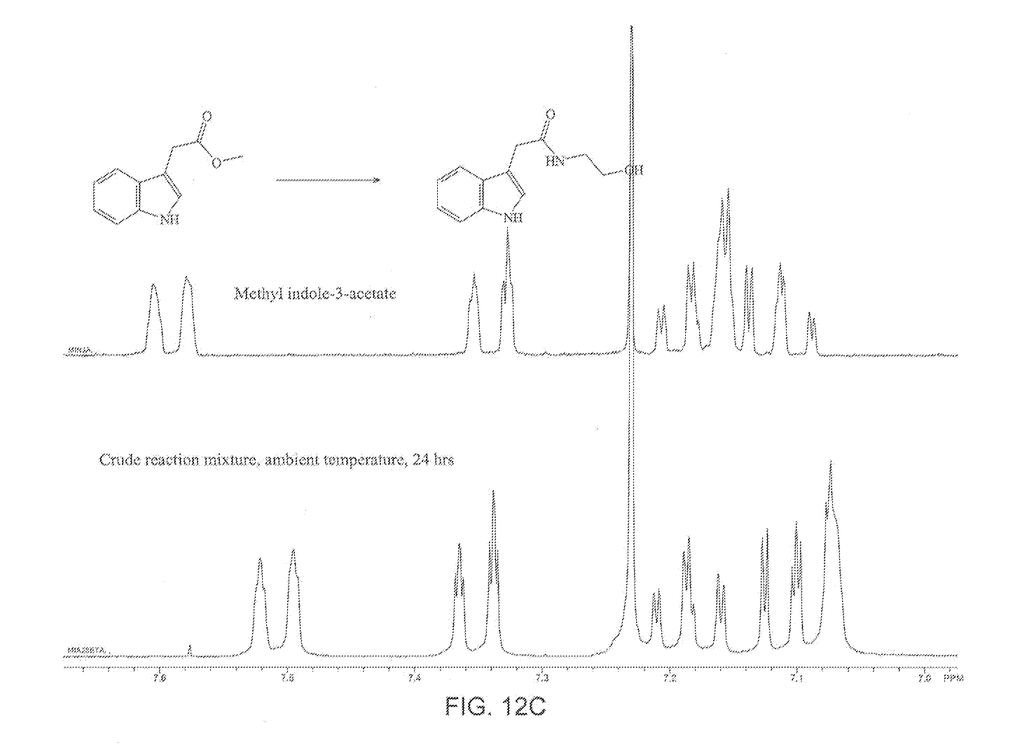

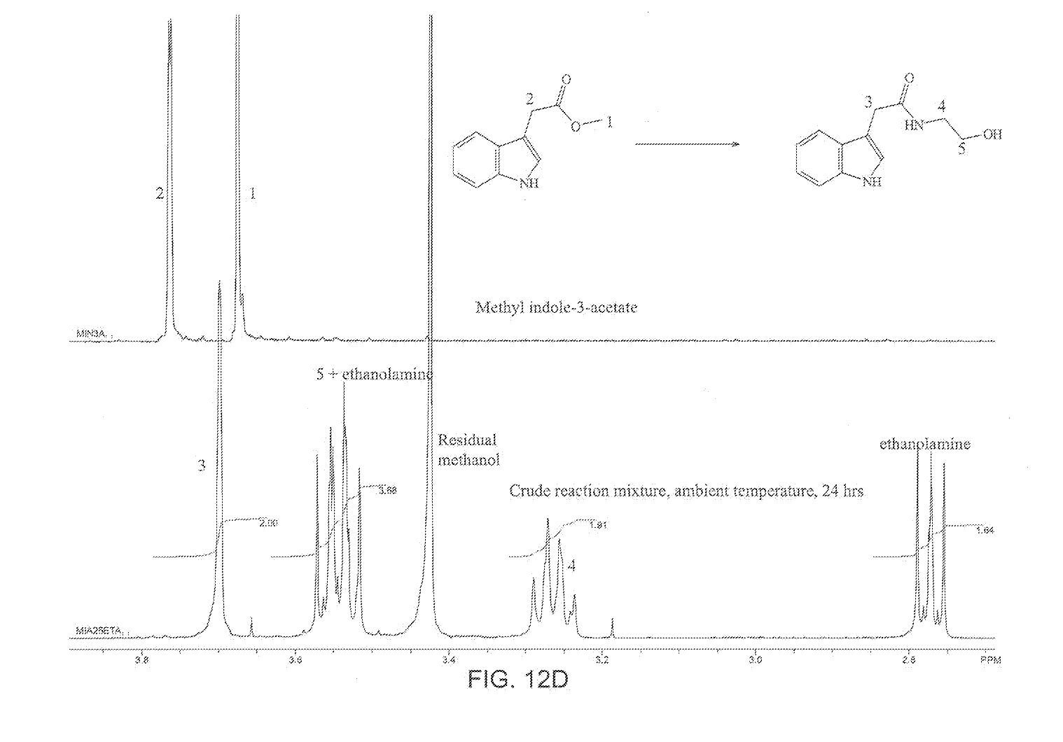

NMR spectra for Example 12 are provided in FIGS. 12A, 12B, 12C, and 12D. The spectra indicate complete conversion of the starting material to the desired product under ambient conditions after 24 hrs. Additional byproducts are observed when the reaction is conducted at 60° C. FIGS. 12C and 12D indicate an aromatic region showing no residual starting material after 24 hours under ambient conditions and clean conversion to the desired product.

### Example 13

### Comparative

Methyl jasmonate in the amount of 2.23 mMole was reacted with 3.4 mMole ethanolamine in the presence of methanolic (7 mL of methanol) sodium carbonate (1 gram) under varying sets of conditions: 24 hours at both ambient conditions and under reflux at 60° C. The mixtures were cooled as needed to room temperature, and the sodium carbonate was removed by filtration. Reaction products were obtained by evaporation of methanol under reduced pressure. The ester and expected resulting amide are depicted as follows.

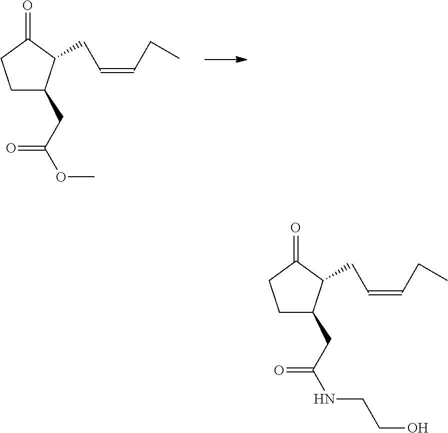

In this reaction, there was no evidence of clean conversion to the desired 2-hydroxyamide derivative at either temperature. Without intending to be bound by theory, it is thought that the presence byproducts could be the result of the amine reacting with the ketone.

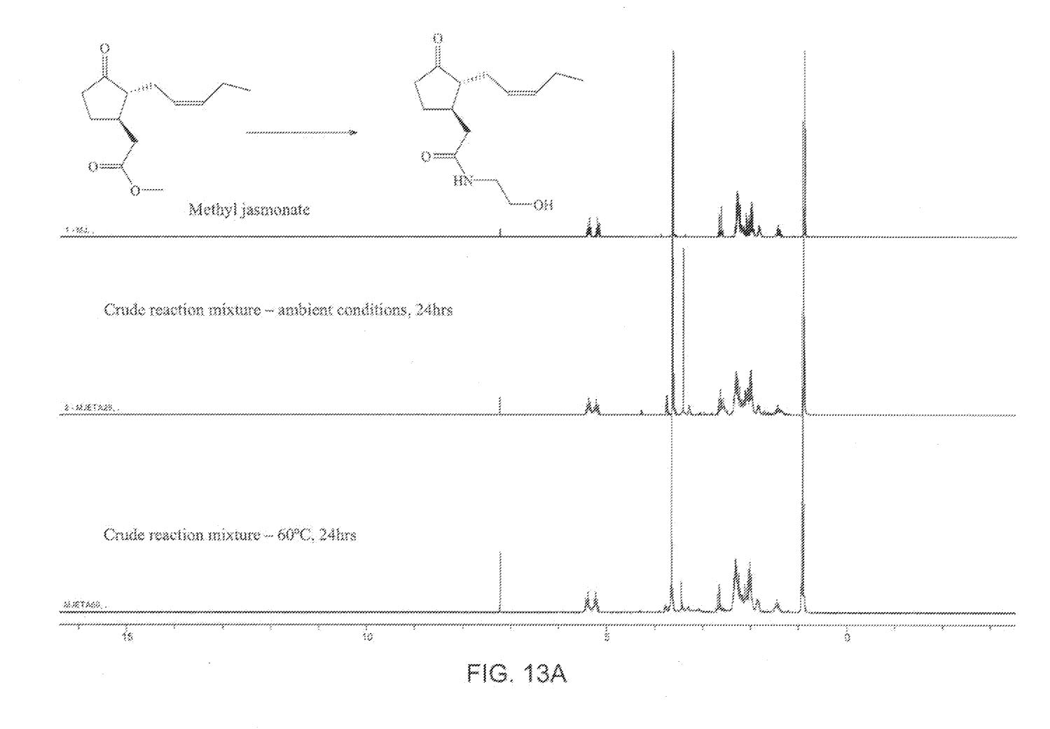

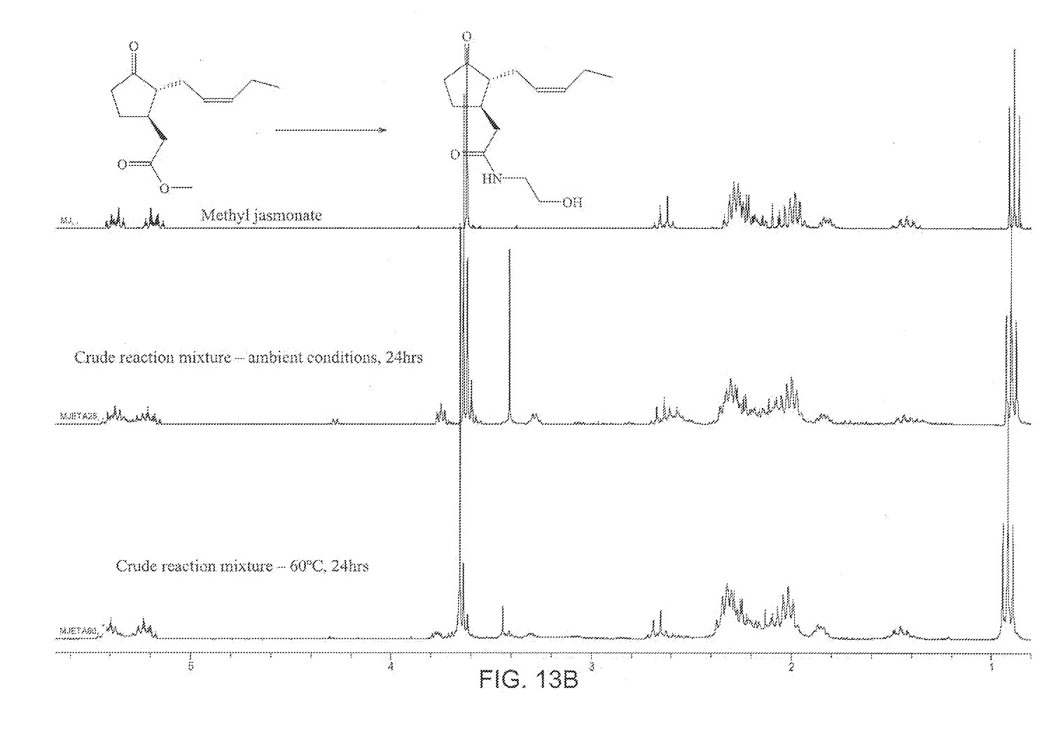

NMR spectra for Example 13 are provided in FIGS. 13A and 13B.

Reference throughout this specification to “one embodiment,” “certain embodiments,” “one or more embodiments” or “an embodiment” means that a particular feature, structure, material, or characteristic described in connection with the embodiment is included in at least one embodiment of the invention. Thus, the appearances of the phrases such as “in one or more embodiments,” “in certain embodiments,” “in one embodiment” or “in an embodiment” in various places throughout this specification are not necessarily referring to the same embodiment of the invention. Furthermore, the particular features, structures, materials, or characteristics may be combined in any suitable manner in one or more embodiments.

Although the invention herein has been described with reference to particular embodiments, it is to be understood that these embodiments are merely illustrative of the principles and applications of the present invention. It will be apparent to those skilled in the art that various modifications and variations can be made to the method and apparatus of the present invention without departing from the spirit and scope of the invention. Thus, it is intended that the present invention include modifications and variations that are within the scope of the appended claims and their equivalents.

1. A process of making hydroxyalkyl amides comprising: reacting an ester with a hydroxyalkyl amine having the formula H₂N—R₃—OH wherein R₃ is a substituted or unsubstituted C2 to C5 alkyl, at a temperature is in the range of about 19° C. to about 80° C. in the presence of a heterogeneous catalyst in an anhydrous solution to form the hydroxyalkyl amides.

2. The process of claim 1, wherein the ester has the formula R₁—CO₂—R₂ wherein R₁ does not have a combination of an amine and a hydroxyl group and R₂ is a substituted or unsubstituted C1 to C10 alkyl group which is not substantially sterically hindered.

3. The process of claim 2, wherein R₁ comprises a hydrocarbon, an alcohol, a carboxylic acid, an ether, a phosphate, a sulfonate, or combinations thereof.

4. The process of claim 1, wherein R₂ comprises a C1 to C5 substituted or unsubstituted alkyl.

5. The process of claim 1, wherein R₂ comprises an unsubstituted C1 to C5 primary alkyl.

6. The process of claim 1, wherein the ester comprises ethyl acetate or the methyl ester of cysteic acid, or combinations thereof.

7. The process of claim 1, wherein the hydroxyalkyl amine comprises 3-aminopropanol, 2-aminoethanol, or combinations thereof.

8. The process of claim 1, wherein the catalyst comprises an alkali metal salt.

9. The process of claim 8, wherein the alkali metal salt comprises a carbonate, an alkoxide, or combinations thereof.

10. The process of claim 8, wherein the alkali metal salt comprises sodium (Na), potassium (K), lithium (Li), cesium (Cs), or combinations thereof.

11. The process of claim 10, wherein the alkali metal salt comprises sodium carbonate, lithium carbonate, or combinations thereof.

12. The process of claim 1 wherein the catalyst comprises N-alkyl ammonium carbonate or N-alkyl ammonium alkoxide.

13. The process of claim 1, wherein the anhydrous solution comprises methanol, ethanol, propanol, or combinations thereof.

14. The process of claim 1, wherein the hydroxyalkyl amides are selected from the group consisting of 3-hydroxypropyl acetamide, 2-hydroxyethyl acetamide, hydroxypropyl and hydroxyethyl amides of cysteic acid and its derivatives, hydroxypropyl and hydroxyethyl amides of ethylbenzoate and its derivatives, hydroxypropyl and hydroxyethyl amides of methyl-5-(3-hydroxyphenyl)-furan-2-carboxylate) and its derivatives, hydroxypropyl and hydroxyethyl amides of methyl indole-3-acetate and its derivatives, or combinations thereof.

15. A method of making monomers suitable for polymeric articles, the method comprising:
providing hydroxyalkyl amides made according to claim 1;
preparing a monomers mixture comprising the hydroxyalkyl amides.

16. The method of claim 15, wherein the hydroxyalkyl amides are selected from the group consisting of 3-hydroxypropyl acetamide, 2-hydroxyethyl acetamide, hydroxypropyl and hydroxyethyl amides of cysteic acid and its derivatives, hydroxypropyl and hydroxyethyl amides of ethylbenzoate and its derivatives, hydroxypropyl and hydroxyethyl amides of methyl-5-(3-hydroxyphenyl)-furan-2-carboxylate) and its derivatives, hydroxypropyl and hydroxyethyl amides of methyl indole-3-acetate and its derivatives, or combinations thereof.

17. The method of claim 15, wherein the alkali metal salt comprises sodium carbonate, lithium carbonate, or combinations thereof.
